(19) United States
(12) Patent Application Publication
(10) Pub. No.: US 2025/0222034 A1
(43) Pub. Date: Jul. 10, 2025

(54) MESENCHYMAL STROMAL CELL-DERIVED EXTRACELLULAR VESICLE-EXOSOMES

(71) Applicant: Biotech Therapeutics, LLC, Estero, FL (US)

(72) Inventors: John Barchilon, Estero, FL (US); Philip W. Askenase, Hamden, CT (US)

(73) Assignee: Biotech Therapeutics, LLC, Estero, FL (US)

(21) Appl. No.: 19/017,125

(22) Filed: Jan. 10, 2025

Related U.S. Application Data
(60) Provisional application No. 63/619,478, filed on Jan. 10, 2024.

(51) Int. Cl. A61K 35/28; C12N 5/00; C12N 5/0775; G01N 33/50

(52) U.S. Cl. CPC A61K 35/28; C12N 5/0018; C12N 5/0662; G01N 33/5023; C12N 2501/15; C12N 2501/2304; C12N 2501/231; G01N 2500/10

(57) ABSTRACT

A method of generating MSC-derived exosome populations may include collecting MSC containing material from living tissue, separating desired mononuclear cells from granulocytes, culturing to multiply the cells, separation of desired cells for further multiplication by washing non-adherent cells and culturing adherent cells, repeating as necessary to obtain a suitably pure population of MSCs, culturing the MSCs in culture media containing negative/healing active cytokines interleukin-4 (IL-4) and interleukin-10 (IL-10) and multifunctional cytokine TGF-ß, and isolating the MSC-derived exosome populations. Diverse MSC-derived exosome populations may be generated by altering the cytokine composition of the culture media. The MSC-derived exosome populations may be screened for effectiveness in treatment of Long Covid using in vitro, in vivo, and pre-clinical testing utilizing model organisms. The exosomes may be administered nasally. Successful MSC-derived exosome populations may be further subjected to patient trials to establish efficacy in treatment of Long Covid via nasal administration of the MSC-derived exosome populations to human subjects. Similar methodologies may be employed to establish efficacy of the MSC-derived exosome populations for treatment of other diseases and conditions related to the central nervous system, spinal cord injury, or neurological diseases, such as Alzheimer disease.

### CROSS-REFERENCE TO RELATED APPLICATIONS

The present application claims the benefit of U.S. Provisional Patent Application No. 63/619,478, filed Jan. 10, 2024, the contents of which are hereby incorporated herein by reference.

### TECHNOLOGY

The present disclosure is related to production of extracellular vesicles and their use to treat medical conditions. More specifically, the present disclosure is directed to production of mesenchymal stromal cell-derived extracellular vesicle populations that are optimized for specific administration routes and treatment of specific medical conditions and use of the same to treat such medical conditions.

### SUMMARY

A system and method of developing mesenchymal stromal cell (MSC) cultures may include directing production of optimized exosome diversity that is targeted for treatment of specific conditions. For example, in one aspect, purified MSCs may be cultured in culture media containing specific combinations of negative acting cytokines to produce MSC-derived exosome populations optimized to treat specific conditions. In one example, purified MSCs may be cultured with one or more negative acting cytokines selected from IL-4, IL-10, and TGF-β to produce therapeutically relevant suppressive/healing mixed MSC-derived exosome populations for nasal administration to treat Long Covid, spinal injuries, neurological disorders, or central nervous system related diseases and conditions. In another aspect, compositions described herein include such MSC cultures, culture media, exosome populations, and pharmaceutical compositions including the MSC-derived exosome populations. In yet another aspect, methods described herein include use of such MSC-derived exosome containing medical compositions to treat medical conditions.

In another aspect, the present disclosure describes a method of developing a MSC culture that produces a mixed exosome population optimized for treatment of a specific medical condition. In one example, the method also includes development of the cell culture to produce a mixed exosome population for use in a drug composition optimized for administration according to a specific route of administration to treat the specific medical condition. In the above or another example, the method may include development of the cell culture to produce a mixed exosome population optimized for use in a specific drug composition to treat the specific medical condition.

In one example, the method includes obtaining or generating MSC-derived exosome populations. The MSC containing material may be collected from living tissue. Desired mononuclear cells may be separated from granulocytes. The cells may be multiplied by culturing. Desired cells may be separated to obtain a suitably pure population of MSCs. The pure MSCs may be cultured in culture media containing a combination of negative acting cytokines comprising one or more of interleukin-4 (IL-4), interleukin-10 (IL-10), or TGF-β to produce a MSC-derived exosome population. Additional pure MSCs populations may be cultured in culture media containing a different combination of negative acting cytokines comprising one or more of interleukin-4 (IL-4), interleukin-10 (IL-10), or TGF-β to produce a MSC-derived exosome population. The resultant diverse MSC-derived exosome populations may be isolated. The isolated MSC-derived exosome populations may be screened for effectiveness in treatment of medical conditions by administration to subjects or models having or designed to test treatments of such medical conditions. Screening may include administration of the MSC-derived exosome populations in different formulations, formats, or administered according to different routes of administration to identify optimal treatment of conditions. In one example, the medical condition comprises a disease and condition related to the central nervous system, spinal cord injury, or neurological diseases, such as Alzheimer disease or Long Covid. In one embodiment, the MSC-derived exosome populations are administered nasally in one or more solid or liquid dosage forms in various strengths. The method may include using the screening to identify the cell culture best targeted to production of a mixed exosome population optimized for treatment of the specific medical condition, which may also include one or more of an optimized formulation, format, or route of administration.

In still another aspect, a method of developing a MSC culture medium targeted to production of a mixed exosome population optimized for treatment of a specific medical condition. In one example, the method also includes development of the cell culture medium to produce the mixed exosome population for use in a drug composition optimized for administration according to a specific route of administration to treat the specific medical condition. In the above or another example, the method may include development of the cell culture medium optimized for use to produce the mixed exosome population for use in a specific drug composition to treat the specific medical condition.

In one example, the method includes obtaining or generating MSC-derived exosome populations. The MSC containing material may be collected from living tissue. Desired mononuclear cells may be separated from granulocytes. The cells may be multiplied by culturing.

Desired cells may be separated to obtain a suitably pure population of MSCs. The pure MSCs may be cultured in culture media containing a combination of negative acting cytokines comprising one or more of interleukin-4 (IL-4), interleukin-10 (IL-10), or TGF-β to produce a MSC-derived exosome population. Additional pure MSCs populations may be cultured in culture media containing a different combination of negative acting cytokines comprising one or more of interleukin-4 (IL-4), interleukin-10 (IL-10), or TGF-β to produce a MSC-derived exosome population. The resultant diverse MSC-derived exosome populations may be isolated. The isolated MSC-derived exosome populations may be screened for effectiveness in treatment of medical conditions by administration to subjects or models having or designed to test treatments of such medical conditions. Screening may include administration of the MSC-derived exosome populations in different formulations, formats, or administered according to different routes of administration to identify optimal treatment conditions. In one example, the medical condition comprises a disease and condition related to the central nervous system, spinal cord injury, or neurological diseases, such as Alzheimer disease or Long Covid. In one embodiment, the MSC-derived exosome populations are administered nasally in one or more solid or liquid dosage forms in various strengths. The method may include using the screening to identify the cell culture medium best targeted to production of a mixed exosome population optimized for treatment of the specific medical condition, which may also include one or more of an optimized formulation, format, or route of administration.

In yet another aspect, a method of developing MSC cultures for production of MSC-derived mixed exosome populations for testing or treatment with respect to a specific disease or condition includes culturing purified populations of MSCs in culture media containing diverse combinations of negative acting cytokines to produce a MSC-derived mixed exosome populations from each purified MSC population. The method further includes isolating the MSC-derived mixed exosome populations produced by the culturing. The method further includes screening the isolated MSC-derived mixed exosome populations to identify one or more of the MSC-derived mixed exosome populations having efficacy in treatment of Long Covid, spinal injury, neurological disorder, or central nervous system related disease or condition. The negative acting cytokines include one or more cytokines selected from IL-4, IL-10, and TGF-β, wherein TGF-β may be present in an amount between 0.01 nM and 100 nM, IL-10 may be present in an amount between about 0.1 pg/ml and 1000 pg/ml, and IL-4 may be present in an amount between 0 pg/ml and 400 pg/ml.

In one example, the screening includes intranasal administration of pharmaceutical compositions including the MSC-derived mixed exosome populations in a dosage including between millions and 10 billion exosomes of a respective MSC-derived mixed exosome population in about 0.5 ml. The intranasal administration may be to the nares, nasal mucosal tissue, or upper respiratory tract and includes administration within a vapor, aerosol, spray, irrigation, or drops.

In the above or another example, the screening includes pre-clinical testing and patient trials and utilizes both in vitro and in vivo techniques.

In one example, the MSC-derived mixed exosome populations are administered to model organisms, human subjects, or both by multiple routes of administration to identify optimal delivery routes.

In any of the above or another example, the screening includes administering pharmaceutical compositions including the MSC-derived mixed exosome populations intravenously, intranasally, intra-arterially, or orally.

In any of the above or another example, the screening includes administering the MSC-derived mixed exosome populations to a model organism. The model organism utilized may exhibit one or more Long Covid pathologies for which the MSC-derived mixed exosome populations are screened. The pathologies may include olfactory disfunction including anosmia or dysgeusia, chronic fatigue, paraesthesia, headache, sleep problems, and cognitive or neurological impairment.

In the above or another example, the screening includes administering the MSC-derived mixed exosome populations directly on tendons, cartilage, or burned or denuded surfaces of skin of human subjects, model organisms, or both.

In one example, the diverse MSC-derived mixed exosome populations are administered in treatments to a model organism to test efficacy of the exosome populations, and the model organism may be a rodent or non-human primate.

In one example, the MSC-derived mixed exosome populations are administered intranasally to the nares, nasal mucosal tissue, or upper respiratory tract of human subjects or model organisms. In a further example, the pharmaceutical composition may be administered within a vapor, aerosol, spray, irrigation, or drops.

In still yet another aspect, a method of identify an exosome population for testing or treatment of with respect to a specific disease or condition may include screening a plurality of diverse MSC-derived mixed exosome populations for efficacy in treatment of a specific disease or condition, each of the MSC-derived mixed exosome populations being isolated from a MSC culture cultured in a culture medium containing a different combination of negative acting cytokines including two or more of IL-4, IL-10, and TGF-β.

In one example, the different combinations of negative acting cytokines include TGF-β in amounts between 0.01 nM and 100 nM, IL-10 in amounts between about 0.1 pg/ml and 1000 pg/ml, and IL-4 in amounts between 0 pg/ml and 400 pg/ml.

In the above or another example, the screening includes pre-clinical testing and patient trials and includes utilizing both in vitro and in vivo techniques.

In any of the above or another example, the screening includes administering the MSC-derived mixed exosome populations to model organisms, human subjects, or both by multiple routes of administration to identify optimal delivery routes.

In one example, the screening includes administering pharmaceutical compositions including the MSC-derived mixed exosome populations intravenously, intranasally, intra-arterially, or orally.

In any of the above or another example, the screening includes administering the MSC-derived mixed exosome populations directly on tendons, cartilage, or burned or denuded surfaces of skin.

In any of the above or another example, the screening includes screening for effective administration route, format, or both to treat the disease or condition.

In any of the above or another example, the specific disease or condition may be marked by immune system derived inflammation, cellular degeneration, or both.

In any of the above or another example, the disease or condition may be Long Covid, a spinal injury, a neurological disorder, or central nervous system related disease or condition.

In a further example, the screening includes evaluation of pharmaceutical compositions including the MSC-derived mixed exosome populations for effectiveness in modulation of one or more of immune response inflammation and mediation of cellular restoration or neuroprotective vascular healing and regenerative effects.

In any of the above or another example, the screening includes intranasal administration of pharmaceutical compositions including the MSC-derived mixed exosome populations in a dosage including between millions and 10 billion exosomes of a respective MSC-derived mixed exosome population in about 0.5 ml.

In one example, results of intranasal administrations are quantitated by clinical neuropsychiatric criteria on a weekly basis.

In any of the above or another example, efficacy for the treatment of the specific disease or condition may be monitored using questionnaires, neuropsychiatric testing, physical testing, physiological testing, pathological testing, or combination thereof.

In any of the above or another example, the screening includes administration of the MSC-derived mixed exosome populations in various dosage amounts and thereafter determining optimal dosing via testing including questionnaires, neuropsychiatric testing, physical testing, physiological testing, pathological testing, or combination thereof.

In any of the above or another example, efficacy may be analyzed by observation or assays of expression levels or presence of relevant mRNA, proteins, disease markers, or combination thereof.

In any of the above or another example, screening includes experimentally determining dosage amount of MSC-derived mixed exosome population to be included in pharmaceutical compositions based on one or more of the specific disease or condition treated, the number of doses to be administered, the route of administration, or characteristics of the subject.

In one example, the MSC-derived mixed exosome populations are administered to a model organism to test effectiveness of the exosome populations on the specific disease or condition. For instance, the model organism utilized may exhibits one or more Long Covid pathologies for which the diverse mixed exosome populations are to be screened selected from olfactory disfunction including anosmia or dysgeusia, chronic fatigue, paraesthesia, headache, sleep problems, or cognitive or neurological impairment. In one example, the model organism exhibits cognitive or neurological impairment established by pathological analysis, neuropsychiatric testing, physical testing, physiological testing, or combination thereof. In a further example, the cognitive or neurological impairment may be established utilizing Morris water maze testing over time.

In any of the above or another example, the method further includes sorting one or more of the MSC-derived mixed exosome populations to generate custom combinations of exosome phenotypes for use in further screening, treatments, or both.

In one example, the MSC-derived mixed exosome populations are administered in treatments to a model organism to test efficacy of the exosome populations on a specific disease or condition, and the model organism may be a rodent or non-human primate.

In one example, the MSC-derived mixed exosome populations are administered intranasally to the nares, nasal mucosal tissue, or upper respiratory tract of human subjects or model organisms. In a further example, the pharmaceutical composition may be administered within a vapor, aerosol, spray, irrigation, or drops.

In one aspect, a method of supplying a pharmaceutical composition or ingredients for producing a pharmaceutical composition for treatment of a specific disease or condition. The method may include providing a MSC-derived mixed exosome population for administration to a subject in a pharmaceutical composition to treat a specific disease or condition, the MSC-derived mixed exosome population may be produced by culturing pure MSCs in culture media including a specific combination of negative acting cytokines, the specific combination of negative acting cytokines identified by screens of a plurality of different MSC-derived mixed exosome populations, each produced by a pure MSC culture cultured in a culture media including a different combination of the negative acting cytokines for efficacy in treating the specific disease or condition. The negative acting cytokines may include two or more of IL-4, IL-10, and TGF-β, and the different combinations of negative acting cytokines may include TGF-β in amounts between 0.01 nM and 100 nM, IL-10 in amounts between about 0.1 pg/ml and 1000 pg/ml, and IL-4 in amounts between 0 pg/ml and 400 pg/ml.

In one example, the specific disease or condition may be marked by immune system derived inflammation, cellular degeneration, or both.

In the above or another example, the disease or condition may be Long Covid, a spinal injury, a neurological disorder, or central nervous system related disease or condition. In a further example, the MSC-derived mixed exosome populations are screened for effectiveness in modulation of immune response inflammation and mediation of cellular restoration. Further to one of the above examples, the MSC-derived mixed exosome populations are screened for effectiveness in neuroprotective vascular healing and regenerative effects.

In one example, the MSC-derived mixed exosome populations are administered to a model organism to test effectiveness of the exosome populations on the specific disease or condition. For instance, the model organism utilized may exhibits one or more Long Covid pathologies for which the diverse mixed exosome populations are to be screened selected from olfactory disfunction including anosmia or dysgeusia, chronic fatigue, paraesthesia, headache, sleep problems, or cognitive or neurological impairment. In one example, the model organism exhibits cognitive or neurological impairment established by pathological analysis, neuropsychiatric testing, physical testing, physiological testing, or combination thereof. In a further example, the cognitive or neurological impairment may be established utilizing Morris water maze testing over time.

In one example, the screening includes evaluation of pharmaceutical compositions including the MSC-derived mixed exosome populations for effectiveness in modulation of immune response inflammation and mediation of cellular restoration.

In one example, the screening includes evaluation of pharmaceutical compositions including the MSC-derived mixed exosome populations for effectiveness in neuroprotective vascular healing and regenerative effects.

In the above or another example, the screening includes administering pharmaceutical compositions including the MSC-derived mixed exosome populations to model organisms, human subjects, or both to identify one or more of the pharmaceutical compositions effective for treatment of the specific disease or condition.

In one example, the diverse MSC-derived mixed exosome populations are administered in treatments to a model organism to test efficacy of the exosome populations on a specific disease or condition, and the model organism may be a rodent or non-human primate.

In any of the above or another example, the MSC-derived mixed exosome populations are administered intranasally to the nares, nasal mucosal tissue, or upper respiratory tract of human subjects or model organisms. In a further example, the pharmaceutical composition may be administered within a vapor, aerosol, spray, irrigation, or drops.

In the above or another example, results of intranasal administrations are quantitated by clinical neuropsychiatric criteria on a weekly basis.

In various embodiments of the above aspects and examples, MSC-derived mixed exosome populations may administered within a pharmaceutically acceptable carrier including an aqueous or nonaqueous solution, dispersion, suspension, or emulsion. In one example, the pharmaceutical composition may be provided in an enriched solution for dilution, suspension, or dispersion just prior to administration.

In various embodiments of the above aspects and examples, the MSC-derived mixed exosome populations may be administrated via parenteral or oral administration, transmucosal or transdermal administration, e.g., intranasal, oral absorption, inhalation, such as pulmonary delivery.

In one example, the pharmaceutical composition may be formulated for injection, e.g., intravenous injection. In the above or another example, MSC-derived mixed exosome populations are administered in a pharmaceutical composition including an aqueous or nonaqueous carrier, diluent, solvent, or vehicle selected from one or more of water, sodium chloride solution, polyols (such as glycerol, propylene glycol, polyethylene glycol), carboxymethylcellulose and suitable mixtures thereof, vegetable oils (such as olive oil), or injectable organic esters such as ethyl oleate. In one example, the pharmaceutical carrier may be solid and includes one or more of lactose, terra alba, sucrose, talc, gelatin, agar, pectin, acacia, magnesium stearate, or stearic acid.

In one example, the pharmaceutical carrier may be liquid and includes one or more of sugar syrup, peanut oil, olive oil, or water.

In various embodiments of the above aspects and examples, the MSC-derived mixed exosome populations are provided or administered in a pharmaceutical composition having an oral dosage form including water, glycols, oils, flavoring agents, preservatives, coloring agents. In a further example, the oral dosage form includes an oral liquid preparation including a suspension, elixir, or solution. In one example, the MSC-derived mixed exosome populations are provided or administered in a pharmaceutical composition including a solid oral dosage form including a carrier selected from starches, sugars, microcrystalline cellulose, diluents, granulating agents, lubricants, binders, disintegrating agents. In one example, the solid oral dosage form includes a powder, capsule, or tablet.

### DESCRIPTION

Mesenchymal stem cells (MSCs) are multipotent stem cells found in bone marrow, adipose tissue, connective tissue, placenta, amniotic fluid, umbilical cord tissue and a few others. MSCs differentiate into numerous cell types including osteoblasts, adipocytes, chondroblasts, myocytes, neurocytes, stromal cells, hepatocytes, cardiomyocytes, endothelial cells, epithelial cells, and pancreatic cells. These cells are of clinical interest due, at least in part, to their roles in immune response suppression and promotion of tissue repair.

Extracellular vesicles (EVs) are lipid bilayer particles released by most cells and include exosomes. Exosomes are nanoparticles comprising a surface lipid bilayer. Exosomes are involved in intercellular communication, including cell differentiation signaling and transport of RNAs, mRNAs, peptides and small proteins. Certain functions of exosomes are those that have been historically attributed to the cells that produce them. Accordingly, exosomes are of therapeutic value for cell based treatments.

Exosomes transmit information to target cells through their contained internal substances to modulate activity or function of the target cells. MSCs generate diverse populations of exosomes characterized by their cargo, which include those that transport various proteins and genetic material, such as, mRNA and miRNA, the identity of which drives the change in activity and function of target cells. In various embodiments, the present description describes exosomes compositions derived from MSCs and administration of pharmaceutical compositions including the exosome compositions to modulate immune response inflammation and mediate cellular restoration. These pharmaceutical compositions including the exosome compositions may also be administered to provide neuroprotective vascular healing, and regenerative effects. The pharmaceutical compositions including the exosome compositions offer an increased safety profile compared to their cellular origins because they are more biocompatible, have low immunogenicity, and do not replicate, thus, alleviating risk of tumorigenesis.

The present disclosure describes methods of generating suppressive/healing mixed exosome populations, culture media for generating exosome populations, exosome compositions including suppressive/healing mixed exosome populations, pharmaceutical compositions comprising suppressive/healing mixed exosome populations, and methods of treating diseases and conditions with suppressive/healing mixed exosome compositions. It is to be understood that the inventive aspects of which are not limited to specific synthetic methods or to particular reagents unless otherwise specified as such may vary. It is also to be understood that the terminology used herein is for the purpose of describing particular aspects only and is not intended to be limiting. Although any methods and materials similar or equivalent to those described herein may be used in the practice or testing of the present invention.

In various embodiments, the present disclosure describes systems and methods for creating growth environments that optimize production of desirable populations of exosomes for treatment of specific diseases and conditions, via specific administration routes, in specific dose formats, or combination thereof. For example, the present disclosure describes systems and methods of culturing MSC cultures comprising unique combinations of negative acting cytokine media to produce optimal suppressive/healing mixed exosome populations for administration in exosome compositions to treat diseases and conditions. In some embodiments, the target exosome populations may be experimentally identified as described herein for the treatment of specific diseases and conditions, via specific administration routes, in specific dose formats, or combination thereof.

The diseases and conditions may include those marked by immune system derived inflammation, cellular degeneration, or both. Some example diseases and conditions may include Long Covid, spinal injuries, neurological disorders, or central nervous system related diseases and conditions.

The exosome compositions may be administered to subjects by various routes, e.g., intravenously, intranasally, intra-arterially, or orally. In some instances, the exosome compositions may be optimized for treatment of a specific disease or condition as well as the route of administration. For example, the systems and methods of culturing MSC cultures comprising unique combinations of negative acting cytokines mixture media may be utilized to produce suppressive/healing mixed exosome populations that are optimized to treat a specific disease or condition when administered via a specific route, such as when administered intranasally. The systems and methods employ good manufacturing practice (GMP) conditions and clinical-grade reagents for preparation of cells and secreted exosome phenotypes expressing MSC markers such as cluster of differentiation (CD) 29, CD44, CD90, CD73 and CD105 that enable MSC exosomes to reside in injured and inflamed tissues and that express common markers of exosomes, such as tetraspanins CD9, CD63, and CD81.

According to one methodology, an MSC culture is exposed to a culture medium including specific combinations of negative acting cytokine mixture to produce an exosome population that is collected and formulated into a pharmaceutical preparation for administration to subjects in a suitable format via a suitable route to treat a disease or condition.

In some embodiments, the methodology may additionally or alternatively include collecting MSCs, isolating MSCs, and generating a MSCs culture of suitable purity. For example, cells including MSCs may be collected by biopsy or aspiration from bone marrow, fatty tissue, umbilical cord, Wharton's jelly, amniotic fluid, placenta, fatty tissue, connective tissues. The collected cells may include a variety of cells in various cell stages. For instance, bone marrow contains red and yellow marrow. Red marrow contains blood stem cells developing into blast cells, reticulocytes, RBCs, lymphocytes, neutrophils, megakaryocytes, and platelets. Yellow marrow contains copious fat and stem cells leading to lipocytes (fatty tissue), chondrocytes (cartilage). osteocytes (bone), and tenocytes (tendons). Exosome populations generated herein from bone marrow for treatment of disease and conditions may be derived from yellow marrow.

In one example, bone marrow samples are aspirated from the posterior iliac of a donor. Donors are preferably healthy and within 16-43 age range. The mononuclear cells may be separated from the sample. Bone marrow mononuclear cells are a mixed population of single nucleus cells including monocytes, lymphocytes, and hematopoietic stem and progenitor cells.

Centrifugation may be used to separate the desired mononuclear cells from granulocytes. For instance, the sample may be separated by density gradient centrifugation in Ficoll® (Tianjin Haoyang Biotech, Inc., Tianjin, China), Percoll®, sucrose, or other suitable gradient medium.

MSCs can be characterized by plastic-adherence under standard culture conditions. Accordingly, plastic adherent mononuclear cell fraction may be cultured according to known techniques. In one example, cells are cultured in multilevel flasks using low-glucose Dulbecco's modified Eagle's medium (DMEM-LG) containing 10% fetal bovine serum and 1% penicillin-streptomycin in a humidified incubator at 37° C. under 5% CO₂. Nonadherent cells are progressively removed. When the plastic adherent, self-replenishing, macrophage depleted, primary MSCs are expanded to 80% confluence, the cells are harvested or further expanded to reach cell counts of enriched MSCs suitable to reach variable concentrations of MSCs for derivation of secreted exosomes. Nonadherent cells may be removed by washing. The medium may be replaced every 3 days or as otherwise determined to be suitable after the initial plating. When well-developed colonies of expanding MSCs appear, the cultures may be trypsinized to release the adherent cells and passaged into a new multi surface flask for further expansion of adherent self-replenishing cells. For example, at about 80-85% confluency, adherent cells (enriched in MSC) may be detached by treatment with 0.125% trypsin and 0.1% EDTA.

MSCs can be characterized by expression or lack thereof of various surface molecules, such as expression of CD105, CD73, and CD90 and lack of expression of CD45, CD34, CD14 or CD11b, CD79a or CD19, and HLA-DR. Accordingly, the resulting detached adherent cells may then be analyzed for purity. Flow cytometry may be employed for surface marker analysis looking for CD73, CD105, CD90, CD29, human leukocyte antigen HLA-DR, CD45, CD34, CD14, and CD79 to verify dominance of adherent self-replenishing. In another example, purity may be established according to adherence to at least the minimum criteria for identification of human MSC proposed by the Mesenchymal and Tissue Stem Cell Committee of the International Society for Cellular Therapy (ISCT): adherence to plastic substrates under standard cultured conditions; at least 95% positive rate of CD105, CD73 and CD90 expression, and ≤2% positive expression rate CD45, CD34, CD14 or CD11b, CD79a or CD19 or HLA-DR (human leukocyte antigen-DR); and ability to induce differentiation into osteoblasts, chondrocytes and adipocytes after induction by standard methods in vitro.

Additional criteria for release of purified MSC cultures for use in exosome production protocols may also include absence of visible clumps seen in light microscopy, spindle-shaped morphology, absence of contamination by: pathogens (as documented by aerobic and anaerobic cultures), virus for hepatitis B surface antigen, hepatitis B core antibody, hepatitis C virus antibody, human immunodeficiency virus and antibodies I and II, cytomegalovirus IgM, and syphilis antibody, which may be determined by enzyme-linked immunosorbent assays (ELISA); and cell viability greater than 95%, which may be determined by trypan blue testing.

Released enriched MSCs may be cultured using various culture media targeted to production of optimal populations of exosome for desired applications. For example, the culture media may incorporate one or more, such as combinations of, negative acting cytokines. In one example, the culture media includes negative acting cytokines comprising one or more of IL-4, IL-10, or TGF-β. In various embodiments, additional cytokines may additionally or alternatively be included.

In various embodiments, cells may be cultured in culture media comprising a negative acting cytokine mixture comprising or consisting of one or more of TGF-β, IL-10, or IL-4. For example, TGF-β may be present in the culture medium an amount between about 0 nM and about 100 nM, such as between about 0.01 nM and about 0.10 nM, between about 0.1 nM and about 0.5 nM, between about 0.5 nM and about 1 nM, between about 1 nM and about 10 nM, between about 10 nM and about 50 nM, between about 10 nM and about 100 nM, greater than or less than about 0.01 nM, greater than or less than about 1 nM, greater than or less than about 10 nM, greater than or less than about 50 nM, greater than or less than about 100 nM. IL-10 may be present in the culture medium in an amount between about 0 pg/ml and about 1000 pg/ml, such as between about 0.1 pg/mL and about 1 pg/mL, between about 1 pg/mL and about 10 pg/mL, between about 10 pg/mL and about 100 pg/mL, between about 100 pg/mL and about 1000 pg/mL, greater than or less than about 0.01 pg/mL, greater than or less than about 1 pg/mL, greater than or less than about 10 pg/mL, greater than or less than about 50 pg/mL, greater than or less than about 100 pg/mL, greater than or less than about 500 pg/mL, greater than or less than about 1000 pg/mL. IL-4 may be present in the culture medium in an amount between about 0 pg/ml and about 400 pg/ml, such as between about 0 pg/ml and about 0.4 pg/ml, between about 0.4 pg/mL and about 1 pg/mL, between about 1 pg/mL and about 10 pg/mL, between about 10 pg/mL and about 100 pg/mL, between about 100 pg/mL and about 400 pg/mL, greater than or less than about 0.4 pg/mL, greater than or less than about 1 pg/mL, greater than or less than about 10 pg/mL, greater than or less than about 40 pg/mL, greater than or less than about 100 pg/mL, greater than or less than about 200 pg/mL, greater than or less than about 300 pg/mL, or greater than or less than about 400 pg/mL.

In various embodiments, culture media comprises IL-4, IL-10, and TGF-β in any combination of the above amounts or ranges thereof. For example, TGF-β may be present in a culture medium in an amount between 0 nM and about 100 nM, IL-10 may be present in the culture medium in an amount between about 0 pg/ml and about 1000 pg/ml, and IL-4 may be present in the culture medium in an amount between about 0 pg/ml and about 400 pg/ml. In one example, TGF-β may be present in a culture medium in an amount between 0.01 nM and about 100 nM, IL-10 may be present in the culture medium in an amount between about 0.1 pg/ml and about 1000 pg/ml, and IL-4 may be present in the culture medium in an amount between about 0.4 pg/ml and about 400 pg/ml.

In one example, MSCs may be cultured in culture media including combinations of negative acting cytokines to identify optimal culture media. For example, culture media may be utilized that include TGF-β in amounts of O nM, 0.01 nM, 0.10 nM, 1.0 nM, 10 nM, and 100 nM, IL-10 in amounts of 0 pg/ml, 0.1 pg/ml, 1.0 pg/ml, 10 pg/ml, 100 pg/ml, and 1000 pg/ml, and IL-4 in amounts of 0 pg/ml, 0.4 pg/ml, 4.0 pg/ml, 40 pg/ml, and 400 pg/ml. Screening of exosome populations produced from MSC cultures cultured in culture media including combinations of the above negative acting cytokines may be used to screen for promising combinations of negative acting cytokines with respect to conditions to be treated. The screening may also include screening with respect to route of administration, dosage forms, formulation, or the like. Promising combinations may be further modified for further screening to optimize the combinations with respect to presence or ratio of the negative acting cytokines.

The methodology may include isolation of exosomes produced by the MSC cultures. The isolated exosomes may be further enriched, characterized or both. For example, exosomes may be isolated by various methods, such as differential ultracentrifugation to about 100,000 g, ultrafiltration, immunoaffinity capture, microfluidics, size-exclusion chromatography, or precipitation. Additionally, harvested MSC-derived exosomes may be enriched further employing antigen or antibody mediated affinity chromatography employing appropriately linked syringe columns. Flow cytometry may be used to characterize exosome phenotypes. The above and additional example methodologies, which may include MSC collection, separation of the collected MSCs, and multiplication of MSCs, may include those described by Nakazaki M, et al. Small extracellular vesicles released by infused mesenchymal stromal cells target M2 macrophages and promote TGF-β upregulation, microvascular stabilization and functional recovery in a rodent model of severe spinal cord injury. J Extracell Vesicles. 2021 September;10(11). PMID: 34478241; Bryniarski K, et al. Free Extracellular miRNA Functionally Targets Cells by Transfecting Exosomes from Their Companion Cells. PLOS One. 2015 Apr. 29;10(4). PMID: 25923429; Jeppesen DK, et al. Reassessment of Exosome Composition. Cell. 2019 Apr. 4;177(2):428-445. PMID: 30951670; Lane RE, et al. Purification Protocols for Extracellular Vesicles. Methods Mol Biol. 2017;1660:111-130. PMID: 28828652; Coughlan C, et al. Exosome Isolation by Ultracentrifugation and Precipitation and Techniques for Downstream Analyses. Curr Protoc Cell Biol. 2020 September;88(1):e110. PMID: 32633898, all of which are hereby incorporated herein by reference.

The particular cytokines or combinations thereof used in culture media may be selected by resulting exosome characterization alone or in combination with use testing on subjects, e.g., pre-clinical model organisms, patient trials. Example conditions treated may include conditions or diseases related to the central nervous system, spinal cord injury, neurological diseases, or Long Covid. In one configuration, the condition is Alzheimer disease.

In one embodiment, a method of identifying relevant suppressive/healing exosome mixed populations for treatment of a specific disease or condition includes exposing an MSC culture to culture media including specific combinations of negative acting cytokines to produce sample suppressive/healing exosome mixed populations. The method may additionally or alternatively include receiving, formulating, or providing the exosome populations for formulating pharmaceutical preparations including the exosome populations. The method may additionally or alternatively include receiving, administering, or providing the pharmaceutical preparations for administration to test subjects. Effectiveness of the treatment may be monitored using questionnaires, neuropsychiatric testing, physical testing, physiological testing, pathological testing, or combination thereof. In various embodiments, the method may include identifying suitable or optimal administration formats, routes, or both to treat the disease or condition. Tests may be targeted to specific dosage formats and/or administration routes to treat a specific condition whereby MSC cultures are cultured in multiple combinations of negative acting cytokines and administered to subject in the same or different format, by the same or different route, or combination thereof to treat a target condition. Indeed, any combination of test variables may be held constant or varied to identify the optimal parameters.

Exosome populations derived from various combinations of culture media including specific combinations of negative acting cytokines may be screened utilizing in vitro and in vivo techniques. As introduced above, exosome populations may be administered to a model organism to test effectiveness of the exosome population on a disease or condition. Example model organisms may include rodents such as mice, rats, or hamsters, non-human mammals, such as non-human primates, or other suitable organisms.

The model organism may be afflicted by or manifest a disease or condition in which the effect of the exosome populations is to be tested. As some diseases and conditions may not be associated with a suitable model organism that may act as a human proxy, a suitably analogous model organism may be used. For example, in one embodiment, a Covid model organism may be used as an analogous model organism to examine exosome population impact of the disease. In some applications, the model organism may manifest specific pathologies of interest with respect to a disease or condition rather than a complete clinical pathology with respect to the disease or condition. For example, Long Covid is a multi-systemic disorder a model organism utilized for pre-clinical testing may exhibit one or more pathologies for which the exosome populations are to be screened, such as olfactory disfunction (anosmia, dysgeusia), chronic fatigue, paraesthesia, headache, sleep problems, or cognitive or neurological impairment. Cognitive or neurological impairment may be established by pathological analysis, neuropsychiatric testing, physical testing, physiological testing, or combination thereof. For example, cognitive or neurological impairment may be established utilizing Morris water maze testing over time.

The model organism may be administered the exosome populations by various routes of administration to identify optimal delivery routes to achieve desired treatment. Effectiveness of the treatment administered to model organisms may be analyzed by observation or assays of expression levels or presence of relevant mRNA, proteins, disease markers or other the like using known molecular techniques.

Suitable exosome population size doses to be included in the pharmaceutical compositions may vary, e.g., based on the disease or condition treated, the number of doses to be administered, the route of administration, characteristics of the subject, e.g., body weight. The optimal exosome population size per dose may be experimentally determined. For example, subjects may be administered various test dosage amounts and thereafter subjected to testing, such as utilizing questionnaires, neuropsychiatric testing, physical testing, physiological testing, pathological testing, or combination thereof, to determine optimal dosing. Dosage testing may include administration to model organism subjects in full and fractional dosages followed by relevant pathological testing, e.g., in a manner similar to that described by Nakazaki M, et al. Small extracellular vesicles released by infused mesenchymal stromal cells target M2 macrophages and promote TGF-β upregulation, microvascular stabilization and functional recovery in a rodent model of severe spinal cord injury. J Extracell Vesicles. 2021 September;10(11). PMID: 34478241, which is hereby incorporated by reference. Subjects may be monitored over time while receiving test dosages at specified intervals to improve optimal dosing size. Dosing size may include populations in the millions or billions of exosomes, for example.

In one example, MSCs may be cultured in culture media including various concentrations, combinations, ratios, or combination thereof of negative acting cytokines comprising IL-4, IL-10, or TGF-β with the goal of producing optimized suppressive/healing mixed exosome population to treat a specified condition when administered to a subject nasally. Nasal administration may include administration of exosome populations within various dosage forms. In one example, the exosome population is within a saline solution or suspension. In one example, the composition may be subjected to pre-clinical screening utilizing suitable in vitro and in vivo testing. For example, various exosome populations may be nasally administered to model organisms to identify effective populations for further study. Identified populations may be further subjected to clinical trials utilizing human subjects. In the above testing, the composition may be administered intra nasally in the range of doses of, e.g., 10 to the 10 (ten billion in about 0.5 ml). The dosage may be based on various factors, such as body weight of prospective subjects. The composition may be administered to the nares, nasal mucosal tissue, or upper respiratory tract. The composition may be administered to subjects nasally, for example, within a vapor, aerosol, spray, irrigation, or drops. According to one configuration, the composition is administered via an inhaler, such as a metered inhaler. Determination of optimum exosome population may vary by dosage form, administration site, or combination thereof for various exosome populations and the efficacy of the exosome populations with respect to treatment of the condition may be determined by methods known in the art. In one embodiment, the condition to be treated is selected from Long Covid, spinal injury, and central nervous system condition.

In another example, culture media is selected to generate diverse suppressive/healing exosome mixed populations. The populations may be sorted to generate custom combinations of exosome phenotypes for use in testing and treatments, such as those described herein, which may include in vitro, in vivo, model organism, and patient trials. The custom combinations may be enriched in one or more exosome phenotypes thereby creating exotic exosome treatments, unparalleled in nature.

According to various embodiments, preparation methodologies for deriving populations of suppressive/healing mixed exosomes from MSCs includes collection, separation/multiplication, and characterization/purification. It will be appreciated that all or portions of the steps may be performed by different parties. Subtle variations in the preparation methodologies can have wide-ranging effects on resulting populations of suppressive/healing exosome. Thus, tests described herein should be utilized to determine optimal populations of suppressive/healing mixed exosomes to treat specific conditions.

Collection may include collecting cells including MSCs from certain living tissues, e.g., bone marrow, fatty tissue, connective tissue, umbilical cord, Wharton's jelly, placenta, or amniotic fluid, utilizing suitable methodology, such as those described herein, e.g., biopsied, scraped, or aspirated.

Separation/multiplication may include separation of desired mononuclear cells from granulocytes utilizing a suitable methodology, such as those described or referenced herein. For example, mononuclear cells may be separated from granulocytes by centrifugation and followed by density gradient centrifugation in Ficoll, Percoll, sucrose or other gradient medium. The cells may be multiplied by culture in suitable containers, such as plastic flasks, with suitable growth media, such as low-glucose Dulbecco's modified Eagle's medium (DMEM-LG) containing 10% fetal bovine serum (FBS) and 1% penicillin-streptomycin in a humidified incubator at 37° C. under 5% CO2. Further separation of desired cells for further multiplication may include washing away non-adherent cells. Multiplication may further include culture of adherent cells in the plastic flasks, removal of adherent cells from the plastic flasks by trypsinization. This may be followed by a second passage onto flasks, with third and sometimes fourth, or additional, passages, each including trypsinization to free up the adherent cells. As described above, about 80% of the flask may be covered by MSCs. The cells may be cultured in a culture medium comprising negative active cytokines. In one example, the culture medium may be otherwise similar to the growth medium used to expand the MSCs or other growth medium suitable for providing an environment favorable for producing exosomes. In a further example, the culture medium may exclude fetal bovine serum all together or following initial cultures or within the final 24 to 48 hours of culturing as to avoid contamination of the exosome the desired exosome population in the fetal bovine serum.

Characterization/purification may include isolation of exosomes produced by the MSCs. The exosomes may be harvested from the culture via differential centrifugation to 100,000 g, ultra-filtration, immunoaffinity capture, microfluidics, size-exclusion chromatography, precipitation, or combination thereof. In one example, antibody washing is used exclude undesired contents. In one embodiment, harvested MSC-derived exosomes are enriched further by antigen-or antibody-mediated affinity chromatography using linked syringe columns. The exosomes comprise a mixed population of suppressive mixed exosomes loaded with cargo aimed at changing target cells in various desirable ways. The cargo is at least partially related to the culture medium, or, more specifically, the representation of respective negative/suppressive acting cytokines present in culture medium in which the MSCs were cultured.

As introduced above, pharmaceutical compositions including the suppressive/healing mixed exosome populations described herein may be formulated in various formats. For example, pharmaceutical compositions including suppressive/healing mixed exosome populations may include the suppressive/healing mixed exosome populations within a pharmaceutically acceptable carrier. The carrier may be an aqueous or nonaqueous solution, dispersion, suspension, or emulsion. In one example, the pharmaceutical composition is provided in an enriched solution for dilution, suspension, or dispersion just prior to administration. The pharmaceutical composition may typically comprise a format corresponding to the carrier. The pharmaceutical composition may be formulated for administration via parenteral or oral administration. The pharmaceutical composition may be formulated for transmucosal or transdermal administration, e.g., intranasal, oral absorption, inhalation, such as pulmonary delivery. The pharmaceutical composition may be formulated for injection, e.g., intravenous injection. Examples of suitable aqueous and nonaqueous carriers, diluents, solvents, or vehicles may include water, sodium chloride solution, polyols (such as glycerol, propylene glycol, polyethylene glycol and the like), carboxymethylcellulose and suitable mixtures thereof, vegetable oils (such as olive oil) and injectable organic esters such as ethyl oleate. In an example, a pharmaceutical carrier employed may be a solid or liquid. Examples of solid carriers may include one or more of lactose, terra alba, sucrose, talc, gelatin, agar, pectin, acacia, magnesium stearate, and stearic acid. In an example, examples of liquid carriers may include one or more of sugar syrup, peanut oil, olive oil, and water. In preparing a disclosed composition in an oral dosage form, any convenient pharmaceutical media can be employed. For example, water, glycols, oils, flavoring agents, preservatives, coloring agents or the like can be used to form oral liquid preparations such as suspensions, elixirs and solutions. Carriers such as starches, sugars, microcrystalline cellulose, diluents, granulating agents, lubricants, binders, disintegrating agents, or the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid or the like. It can also be desirable to include isotonic agents such as sugars, sodium chloride and the like. Prolonged absorption of an injectable pharmaceutical dosage form can be brought about by the inclusion of agents, such as aluminum monostearate and gelatin, which delay absorption. Injectable depot forms may be made by forming microencapsule matrices of the drug in biodegradable polymers such as polylactide-polyglycolide, poly (orthoesters) or poly (anhydrides). Injectable formulations can be sterilized, for example, by filtration through a bacterial-retaining filter or by incorporating sterilizing agents in the form of sterile solid compositions which can be dissolved or dispersed in sterile water or other sterile injectable media just prior to use.

As introduced above, suppressive/healing mixed exosome populations may be used in pre-clinical testing and patient trials to establish efficacy of the treatment and identify optimized populations of suppressive/healing mixed exosomes. Exosome treatment trials are conducted in compliance with current good clinical practice standards and in accordance with the principles set forth under the Declaration of Helsinki (1989). In one example, MSC exosomes are administered to subjects in test doses, e.g., hundreds of millions of exosomes, delivered intravenously, intranasally, intra-arterially, or orally. Intravenous infusion may be performed at a rate of 2 mL/min. The exosomes may be in suitable medium, such as sterile water, sterile sodium chloride 0.9%, water for irrigation, sodium chloride 0.9% irrigation, water for injection, or sodium chloride 0.9% injection. In another example, exosomes are administered directly on tendons, cartilage, burned or denuded surfaces of the skin. Local administration may be diluted, e.g., 100:1 of the stock in billions of exosomes per mL, in suitable diluent, such as normal saline. If the doses of harvested exosomes are frozen, e.g., at −80° F., slow product thawing and then dilution generally is undertaken immediately before administration. Patients and controls may receive doses of MSC-derived exosomes for a total of four doses administered on day 0, day 7, day 14, and day 21. In the case of treatment of patients with Long Covid, results of nasal treatments may be quantitated by clinical neuropsychiatric criteria on a weekly basis.

### Definitions

As used herein, the term “subject” refers to the target of administration, e.g., a human being. In one example, a subject is a human patient. The term does not denote a particular age or sex, and thus, adult and child subjects, as well as fetuses, whether male or female, are intended to be covered. In some embodiments, “subject” may refer to non-human animals to be treated, which may include model organisms.

In various embodiments, a subject may have, be suspected to have, or be at risk of having a disease or condition for which the present compositions and methods may be used to treat. In an example, a subject can or be suspected to have a disease or condition associated with immune system derived inflammation. In an example, a subject can or be suspected to have a disease or condition associated with cellular degeneration. In an example, a subject can or be suspected to have a disease or condition associated with neurological cellular degeneration. In an example, a subject can or be suspected to have a disease or condition associated with a spinal injury. In an example, a subject can or be suspected to have a disease or condition associated with a neurological disorders. In an example, a subject can or be suspected to have a disease or condition associated with the central nervous system. In an example, a subject may be at risk of developing a disease or condition associated with one or more of an immune system derived inflammation, cellular degeneration, neurological cellular degeneration, a spinal injury, a neurological disorder, or the central nervous system. In one example, a subject can have a coronavirus infection, have previously had a SARS-COV-2 infection, be suspected of, e.g., present symptoms, or diagnosed with suffering from Long Covid following a SARS-COV-2 infection, or be at risk of developing Long Covid following a SARS-COV-2 infection.

As used herein, the term “diagnosed” means having been subjected to a physical examination by a person of skill, for example, a physician, and found to have a disease or condition that can be diagnosed or treated by one or more of the disclosed compositions, which may include a pharmaceutical preparation comprising one or more disclosed compositions to a subject, and/or disclosed methods. “Suspected” of having a disease or condition can mean having been subjected to a physical examination by a person of skill, for example, a physician, and found to have a condition that can likely be treated by one or more of the disclosed compositions, which may include a pharmaceutical preparation comprising one or more disclosed compositions to a subject, and/or disclosed methods.

The words “treat” or “treating” or “treatment” refer to therapeutic or medical treatment wherein the object is to slow down (lessen), ameliorate, and/or diminish an undesired physiological change, pathological change, progression, or symptoms with respect to a disease or condition in a subject. “Treat” or “treating” or “treatment” may also refer to causing regeneration or reversal of undesired physiological and/or pathological change with respect to a disease or condition in a subject. As used herein, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Treatment may not necessarily result in the complete recovery or alleviation of symptoms but may reduce or minimize impact and/or progression of the disease or condition. The success of treatments may be monitored by physical examination, pathological examination, or other suitable examination of a subject. The words “treat” or “treating” or “treatment” include palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various examples, the term covers any treatment of a subject, including a mammal (e.g., a human), and includes: (i) preventing the undesired disease or condition, e.g., physiological change, disease, pathological condition, or disorder, from occurring in a subject that can be predisposed but has not yet been diagnosed as having it; (ii) inhibiting the physiological change, disease, pathological condition, or disorder, i.e., arresting its development; or (iii) relieving the physiological change, disease, pathological condition, or disorder, i.e., causing regression of the disease. For example, in an example, treating a disease or condition can reduce the severity in a subject by 1%- 100% as compared to a control (such as, for example, without treatment or relative to another treatment). In an example, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity. For example, treating can reduce one or more symptoms in a subject by 1%- 100% as compared to a control (such as, for example, sleep problems, memory, headaches, mental clarity). It is understood that treatment does not necessarily refer to a cure or complete ablation or eradication of the disease or condition. However, in an example, treatment can refer to a cure or complete ablation or eradication of the disease or condition.

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. The words “prevent” and “preventing” and “prevention” also refer to prophylactic or preventative measures for protecting or precluding a subject (e.g., an individual) not having the given disease or condition or not experiencing or showing significant physiological and/or pathological symptom or complication of a given disease or condition from progressing to that symptom or complication.

As used herein, the terms “administering” and “administration” refer to any method of providing one or more of the disclosed compositions and/or pharmaceutical preparation comprising one or more disclosed compositions to a subject. Such methods are well known to those skilled in the art and include, but are not limited to, the following: oral administration, transdermal administration, administration by inhalation, nasal administration, topical administration, intravaginal administration, ophthalmic administration, intra-aural administration, otic administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent.

In various embodiments, one or more of the disclosed compositions and/or a pharmaceutical preparation comprising one or more disclosed compositions including mixed exosomes can be administered therapeutically; that is, administered to treat an existing disease or condition. In further various examples, one or more of the disclosed compositions and/or a pharmaceutical preparation comprising one or more disclosed compounds can be administered prophylactically; that is, administered for prevention of a disease or condition. In an example, the skilled person can determine an efficacious dose, an efficacious schedule, and an efficacious route of administration for one or more of the disclosed compounds and/or a pharmaceutical preparation comprising one or more disclosed compositions so as to treat or prevent the disease, condition, or manifestations thereof. In an example, the skilled person can also alter, change, or modify an example of an administering step to improve efficacy of one or more of the disclosed compositions and/or a pharmaceutical preparation comprising one or more disclosed compounds.

Disclosed methods may be modified by modifying or changing one or more features or examples of one or more steps of the disclosed methods. That is, those having skill in the art will appreciate upon reading the present disclosure that the methods described herein may be modified while not departing from the teachings herein. The present disclosure is intended to include and encompass such modifications to the methods described herein. For example, a method can be altered by changing the amount of one or more of the disclosed compositions with respect to culture media, target exosome population, or both. A method, for instance, may be changed by changing the amount of one or more negative acting cytokines present in a culture media, changing a ratio of one or more negative acting cytokines present in a culture media, or changing a target exosome population. For example, a method can be altered by changing the amount of one or more of the disclosed compositions, a pharmaceutical preparation comprising one or more disclosed compositions administered to a subject, changing the frequency of administration of one or more of the disclosed compounds and/or a pharmaceutical preparation comprising one or more disclosed compositions to a subject, or changing the duration of time one or more of the disclosed compounds and/or a pharmaceutical preparation comprising one or more disclosed compositions are administered to a subject.

As used herein, “effective amount” and “amount effective” can refer to an amount that is sufficient to achieve the desired result such as, for example, the treatment and/or prevention of a disease or condition or suspected disease or condition. As used herein, the terms “effective amount” and “amount effective” can refer to an amount that is sufficient to achieve the desired an effect on an undesired condition. For example, a “therapeutically effective amount” refers to an amount that is sufficient to achieve the desired therapeutic result or to have an effect on undesired symptoms, but is generally insufficient to cause adverse side effects. In an example, “therapeutically effective amount” means an amount of a disclosed composition that (i) treats the particular disease or condition, (ii) attenuates, ameliorates, or eliminates one or more symptoms of the particular disease or condition, or (iii) delays the onset of one or more symptoms of the particular disease or condition described herein. The specific therapeutically effective dose level for any particular patient will depend upon a variety of factors including the disorder being treated and the severity of the disorder; the specific disclosed compositions and/or a pharmaceutical preparation comprising one or more disclosed compositions, or methods employed; the age, body weight, general health, sex and diet of the patient; the time of administration; the route of administration; the rate of excretion of the disclosed compositions and/or a pharmaceutical preparation comprising one or more disclosed compositions employed; the duration of the treatment; drugs used in combination or coincidental with a disclosed compositions and/or a pharmaceutical preparation comprising one or more disclosed compositions employed, and other like factors well known in the medical arts. For example, it is well within the skill of the art to start doses of a disclosed composition and/or a pharmaceutical preparation comprising one or more disclosed composition at levels lower than those required to achieve the desired therapeutic effect and to gradually increase the dosage until the desired effect is achieved. If desired, then the effective daily dose can be divided into multiple doses for purposes of administration. Consequently, a single dose of a disclosed compositions and/or a pharmaceutical preparation comprising one or more disclosed compositions, or methods can contain such amounts or submultiples thereof to make up the daily dose. The dosage can be adjusted by the individual physician in the event of any contraindications. Dosage can vary, and can be administered in one or more dose administrations daily, for one or several days.

This disclosure describes inventive concepts with reference to specific examples. However, the intent is to cover all modifications, equivalents, and alternatives of the inventive concepts that are consistent with this disclosure. As used in the specification and the appended claims, the singular forms “a”, “an”, and “the” include plural referents unless the context clearly dictates otherwise. As used herein, when referring to any numerical value, the term “about” means a value falling within a range that is +10% of the stated value. Ranges can be expressed herein as from “about” one particular value, and/or to “about” another particular value. When such a range is expressed, a further example includes from the one particular value and/or to the other particular value. Similarly, when values are expressed as approximations, by use of the antecedent “about,” it will be understood that the particular value forms a further example. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

What is claimed is:

1. A method of developing mesenchymal stromal cell (MSC) cultures for production of MSC-derived mixed exosome populations for testing or treatment with respect to a specific disease or condition, the method comprising:
culturing purified populations of MSCs in culture media containing diverse combinations of negative acting cytokines to produce a MSC-derived mixed exosome populations from each purified MSC population;
isolating the MSC-derived mixed exosome populations produced by the culturing; and
screening the isolated MSC-derived mixed exosome populations to identify one or more of the MSC-derived mixed exosome populations having efficacy in treatment of Long Covid, spinal injury, neurological disorder, or central nervous system related disease or condition, wherein the negative acting cytokines comprise IL-4, IL-10, and TGF-β, and wherein TGF-β is present in an amount between 0.01 nM and 100 nM, IL-10 is present in an amount between about 0.1 pg/ml and 1000 pg/ml, and IL-4 is present in an amount between 0 pg/ml and 400 pg/ml.

2. The method of claim 1, wherein the screening comprises intranasal administration of pharmaceutical compositions including the MSC-derived mixed exosome populations in a dosage including between millions and 10 billion exosomes of a respective MSC-derived mixed exosome population in about 0.5 ml, and wherein the intranasal administration is to the nares, nasal mucosal tissue, or upper respiratory tract and comprises administration within a vapor, aerosol, spray, irrigation, or drops.

3. The method of claim 2, wherein the screening comprises pre-clinical testing and patient trials and includes utilizing both in vitro and in vivo techniques.

4. The method of claim 1, wherein the screening comprises administering the MSC-derived mixed exosome populations to model organisms, human subjects, or both by multiple routes of administration to identify optimal delivery routes.

5. The method of claim 1, wherein the screening comprises administering pharmaceutical compositions including the MSC-derived mixed exosome populations intravenously, intranasally, intra-arterially, or orally.

6. The method of claim 1, wherein the screening comprises administering the MSC-derived mixed exosome populations to a model organism, and wherein the model organism utilized exhibits one or more Long Covid pathologies for which the MSC-derived mixed exosome populations are screened, the pathologies selected from olfactory disfunction comprising anosmia or dysgeusia, chronic fatigue, paraesthesia, headache, sleep problems, and cognitive or neurological impairment.

7. The method of claim 1, wherein the screening comprises administering the MSC-derived mixed exosome populations directly on tendons, cartilage, or burned or denuded surfaces of skin of human subjects, model organisms, or both.

8. A method of identify an exosome population for testing or treatment of with respect to a specific disease or condition, the method comprising:
screening a plurality of diverse mesenchymal stromal cell (MSC)-derived mixed exosome populations for efficacy in treatment of a specific disease or condition, wherein each of the MSC-derived mixed exosome populations was isolated from a MSC culture cultured in a culture medium containing a different combination of negative acting cytokines, wherein the negative acting cytokines comprise IL-4, IL-10, and TGF-β.

9. The method of claim 8, wherein the different combinations of negative acting cytokines comprise TGF-β in amounts between 0.01 nM and 100 nM, IL-10 in amounts between about 0.1 pg/ml and 1000 pg/ml, and IL-4 in amounts between 0 pg/ml and 400 pg/ml.

10. The method of claim 8, wherein the screening comprises pre-clinical testing and patient trials and includes utilizing both in vitro and in vivo techniques.

11. The method of claim 8, wherein the screening comprises administering the MSC-derived mixed exosome populations to model organisms, human subjects, or both by multiple routes of administration to identify optimal delivery routes.

12. The method of claim 11, wherein the screening comprises administering pharmaceutical compositions including the MSC-derived mixed exosome populations intravenously, intranasally, intra-arterially, or orally.

13. The method of claim 8, wherein the screening comprises administering the MSC-derived mixed exosome populations directly on tendons, cartilage, or burned or denuded surfaces of skin.

14. The method of claim 8, wherein the screening comprises screening for effective administration route, format, or both to treat the disease or condition.

15. The method of claim 8, wherein the specific disease or condition is marked by immune system derived inflammation, cellular degeneration, or both.

16. The method of claim 8, wherein the disease or condition is Long Covid, a spinal injury, a neurological disorder, or central nervous system related disease or condition.

17. The method of claim 16, wherein the screening comprises evaluation of pharmaceutical compositions including the MSC-derived mixed exosome populations for effectiveness in modulation of one or more of immune response inflammation and mediation of cellular restoration or neuroprotective vascular healing and regenerative effects.

18. The method of claim 8, wherein the screening comprises intranasal administration of pharmaceutical compositions including the MSC-derived mixed exosome populations in a dosage including between millions and 10 billion exosomes of a respective MSC-derived mixed exosome population in about 0.5 ml.

19. The method of claim 8, wherein efficacy for the treatment of the specific disease or condition is monitored using questionnaires, neuropsychiatric testing, physical testing, physiological testing, pathological testing, or combination thereof.

20. The method of claim 8, wherein the screening comprises administration of the MSC-derived mixed exosome populations in various dosage amounts and thereafter determining optimal dosing via testing comprising questionnaires, neuropsychiatric testing, physical testing, physiological testing, pathological testing, or combination thereof.

21. The method of claim 8, wherein efficacy is analyzed by observation or assays of expression levels or presence of relevant mRNA, proteins, disease markers, or combination thereof.

22. The method of claim 8, wherein screening includes experimentally determining dosage amount of MSC-derived mixed exosome population to be included in pharmaceutical compositions based on one or more of the specific disease or condition treated, the number of doses to be administered, the route of administration, or characteristics of the subject.

23. The method of claim 8, wherein the method further comprises sorting one or more of the MSC-derived mixed exosome populations to generate custom combinations of exosome phenotypes for use in further screening, treatments, or both.

24. A method of supplying a pharmaceutical composition or ingredients for producing a pharmaceutical composition for treatment of a specific disease or condition, the method comprising:
providing a mesenchymal stromal cell (MSC)-derived mixed exosome population for administration to a subject in a pharmaceutical composition to treat a specific disease or condition, wherein the MSC-derived mixed exosome population is produced by culturing purified MSCs in culture media including a specific combination of negative acting cytokines, the specific combination of negative acting cytokines identified by screens of a plurality of different MSC-derived mixed exosome populations, each produced by a purified MSC culture cultured in a culture media including a different combination of the negative acting cytokines for efficacy in treating the specific disease or condition, wherein the negative acting cytokines comprise IL-4, IL-10, and TGF-β, and wherein the different combinations of negative acting cytokines comprise TGF-β in amounts between 0.01 nM and 100 nM, IL-10 in amounts between about 0.1 pg/ml and 1000 pg/ml, and IL-4 in amounts between 0 pg/ml and 400 pg/ml.

25. The method of claim 24, wherein the specific disease or condition is marked by immune system derived inflammation, cellular degeneration, or both.

26. The method of claim 24, wherein the disease or condition is Long Covid, a spinal injury, a neurological disorder, or central nervous system related disease or condition.

27. The method of claim 26, wherein the MSC-derived mixed exosome populations are screened for effectiveness in modulation of immune response inflammation and mediation of cellular restoration.

28. The method of claim 26, wherein the MSC-derived mixed exosome populations are screened for effectiveness in neuroprotective vascular healing and regenerative effects.
